(19) United States
(12) Patent Application Publication
(10) Pub. No.: US 2006/0276733 A1
(43) Pub. Date: Dec. 7, 2006

(54) Lim protection system

(76) Inventor: Andrew James Evans, Pymble (AU)

Correspondence Address: KNOBBE MARTENS OLSON & BEAR LLP, 2040 MAIN STREET, FOURTEENTH FLOOR, IRVINE, CA, 92614, US

(21) Appl. No.: 10/563,547

(22) PCT Filed: Jul. 5, 2004
(86) PCT No.: PCT/AU04/00903

§ 371 Date: Jun. 12, 2006

(30) Foreign Application Priority Data
Jul. 4, 2003 (AU) 2003903424

(51) Int. Cl. A61F 5/00

(52) U.S. Cl. 602/3

(57) ABSTRACT

The present invention provides a limb protection system including an enclosure for enclosing the limb section. The invention also includes at least one flexible cuff which, in use, may be appropriately sized to stretch around and compress against parts of the user's limb extending into the limb protection system.

### FIELD OF THE INVENTION

The present invention relates to improvements in a limb protection system for protecting a section of a user's limb against the ingress of moisture, fluids such as water, and unintentional contact such as “bumping”. The invention will be described primarily with reference to its use in medical/treatment applications, but it should be appreciated that the invention can be employed wherever it is necessary or desirable to protect a section of a user's limb against moisture, contact or the like. In addition, when the term “limb” is used herein, it is not simply intended to be limited to legs and arms of a user, but conceivably can extend to the torso etc., of the user as appropriate, and not just human users, but animal users.

### BACKGROUND OF THE INVENTION

In medical treatment, it is often necessary or important to isolate a certain section of a limb of a user from the remainder of the body of user, to prevent it from contacting certain liquids (especially water), solvents, surfaces, etc. For example, where the skin of a user's limb has been cut, grazed, burnt, bandaged, plastered, etc., it is often necessary/desirable to prevent that area from being subjected to contact with water, other liquids or even gases.

The applicant has previously filed a PCT Application, No. WO 02/24014 entitled “Limb Protection System”. The reader is referred to this application.

### SUMMARY OF THE INVENTION

In a first aspect, the present invention provides a limb protection device for protecting a section of a user's limb, the device comprising:
an enclosure for enclosing the limb section; and
at least one flexible cuff integral with and away from which the enclosure extends, the cuff defining an opening into the device and having a tapered profile arranged to allow, in use, the selection of an appropriately sized opening into the device, whereby the appropriately sized opening in the cuff is selectable to stretch around and compress against that part of the user's limb extending therethrough, to prevent the ingress of matter into the device immediately at the device opening.

In the prior art, the applicant has disclosed a device that enclosed a limb to prevent matter, such as moisture (especially water) from reaching the limb section. This is highly advantageous in many medical situations (eg. in bathing and showering of patients with wounds, bandages, plaster etc). The invention of the prior art made use of a tight fitting cuff to provide a stronger seal at the user's limb. However, as different users have different sized limbs, there was a need to manufacture the device in a variety of different sizes, each having a different sized cuff. Correspondingly, hospitals and doctor's surgeries were required to stock the device in a variety of sizes, in order to be able to apply the device to a wide range of users. The present invention preferably provides a “one size fits all” device, as the device may simply be cut or torn at an appropriate point along the tapered cuff to produce a cuff which is sized to fit correctly and tightly around the limb of the user.

The cuff may further comprise a series of spaced-apart protrusions extending along the length of the cuff, wherein the protrusions provide structural integrity to the cuff.

The protrusions may be integrally moulded into the cuff.

The protrusions may be of a substantially semi-circular shape.

The cuff may have a width greater than a wall thickness of the enclosure.

In one embodiment, the cuff may be a continuation of the enclosure and may be formed from the same material as the enclosure, optionally having the same thickness as the enclosure.

In an alternative embodiment, the cuff may be formed from a different material, or a more dense form of the same material as the enclosure. In this regard, the cuff may be formed from a resilient polymeric material such as latex, elastic impregnated plastic, etc to enable its stretching during fitting and removal of the device to a limb.

The enclosure may be a sleeve that is either:
(a) closed at one end, with the cuff located at an opening at the other opposing end; or
(b) open at opposing ends, with respective cuffs located at each end.

In an embodiment with the sleeve in accordance with arrangement (a), the device may be used for isolating entire feet, hands, legs or arms, whereas arrangement (b) may be used for isolating particular sections of limbs (eg. a forearm, wrist, shin, thigh) or for isolating joints (such as the elbow, shoulder, knee or hip).

In an alternative embodiment the sleeve in (a) may be releasably openable at said one end for enabling access to the limb section when the device is fitted to the user (eg. for medical access, or to let air in, or for scratching, adjustments etc). In this regard, a press-seal opening may be employed at said one end.

The sleeve in (a) may be adapted for receiving a user's hand or foot therein; and

The sleeve in (b) may be adapted for receiving the limb right therethrough with the cuff being located on opposing sides of the limb section that is enclosed within the enclosure.

The enclosure and/or the cuff may be transparent, and may be formed from a polymer.

A therapeutic agent may be provided in the interior of the device to aid in the treatment of medical conditions.

### BRIEF DESCRIPTION OF THE DRAWINGS

Notwithstanding any other forms which may fall within the scope of the present invention, preferred forms of the invention will now be described, by way of example only, with reference to the accompanying drawings in which:

FIG. 1 shows a side elevation of a first embodiment of the limb protection device in accordance with the present invention;

FIG. 2 shows a side elevation of a second embodiment of the limb protection device in accordance with the present invention;

FIG. 3 shows in cross sectional side elevation an embodiment of a device, when in use, in accordance with the present invention;

FIG. 4 shows a side elevation of a further alternative device to those shown in FIGS. 1-3;

FIGS. 5 and 6 show, in side elevation, two further alternative limb protection devices for use with a foot/lower leg of a user;

FIGS. 7 and 8 show, in side elevation, two further alternative devices for use with a user's leg;

FIG. 9 shows, in side elevation, another limb protection device in accordance with the present invention; and

FIG. 10 shows, in side elevation, yet a further limb protection device in accordance with the present invention.

### DESCRIPTION OF A SPECIFIC EMBODIMENT

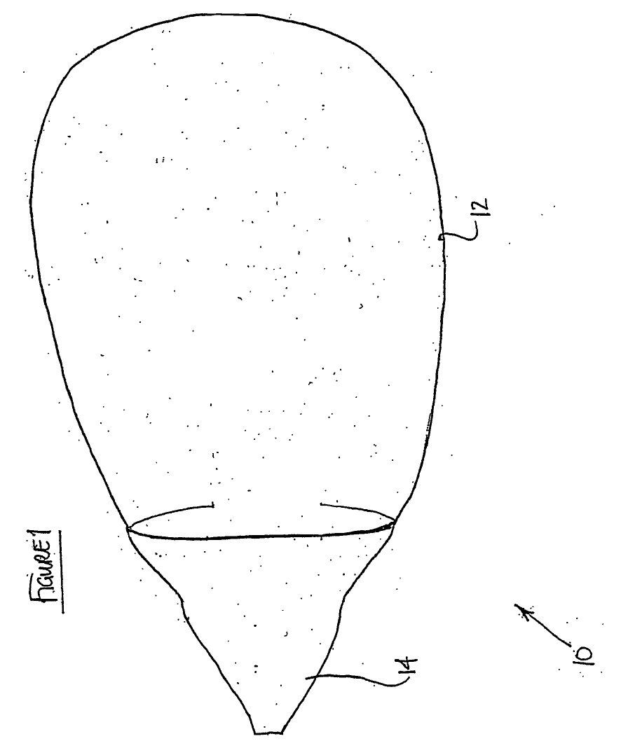

Referring to the drawings, and in particular to FIG. 1, a limb protection device in the form of a protective bag 10 includes a sleeve portion 12 and an integral cuff portion 14. The integral cuff portion has a tapered profile as shown in FIG. 1. The cuff may also, in another embodiment, taper to a “point”. Typically both the sleeve portion and cuff portion are formed from thermoplastic polymers such that they can be moulded integrally together during original formation of the protective bag.

Alternatively, the cuff portion can be formed from a stretchable, resilient polymer, such as a latex or an elastic impregnated polymer to provide a close fit around the user's limb.

Typically the cuff portion tapers from a diameter which is less than the limb of a “small” user, to a diameter which is greater than the limb of a “large” user. This allows the cuff to be sized by allowing the user (or another individual) to cut off or remove a portion of the cuff so that the resultant cuff clasps peripherally around the limb when fitted. It will be understood that the cuff may be sized by an end user, or it may be sized at the point of manufacture. The resultant cuff, once sized, provides a seal to maximise the sealing of the limb against the ingress of matter (especially moisture). It has been observed that not only can the cuff seal against the ingress of any type of liquid, but it can also seal out gas, and lock air into the bag. Thus, the bag can also function like a balloon to protect the section of limb enclosed therein against inadvertent contact, bumping, etc. The seal also prevents the egress of matter from the bag. Therefore, the bag may be filled with ice, with an anti-microbial agent, with a burn treatment cream, or with any other suitable fluid, solid or other therapeutic agent to treat the enclosed limb. The inclusion of the therapeutic agent may preferably allow for more speedy recovery of cuts, bruises, infections, burns, or other ailments.

Furthermore, in one embodiment, the device may be manufactured in sterile conditions and both ends may be sealed, such that the interior of the device remains in a sterile condition until use. If both ends are sealed at manufacture, the interior surface of the device may also be pre-coated with a suitable therapeutic agent prior to sealing.

The embodiment described herein provides a number of advantages, particularly in situations where quick isolation of an injured limb will greatly accelerate the healing process.

For example, when a burn victim is rescued from a fire, the paramedic treating the burn victim may cut an appropriately sized cuff from a device in accordance with an embodiment of the present invention, and immediately apply the device to the patient. This may provide instant relief to the burn victim, while preventing the burn site from becoming infected.

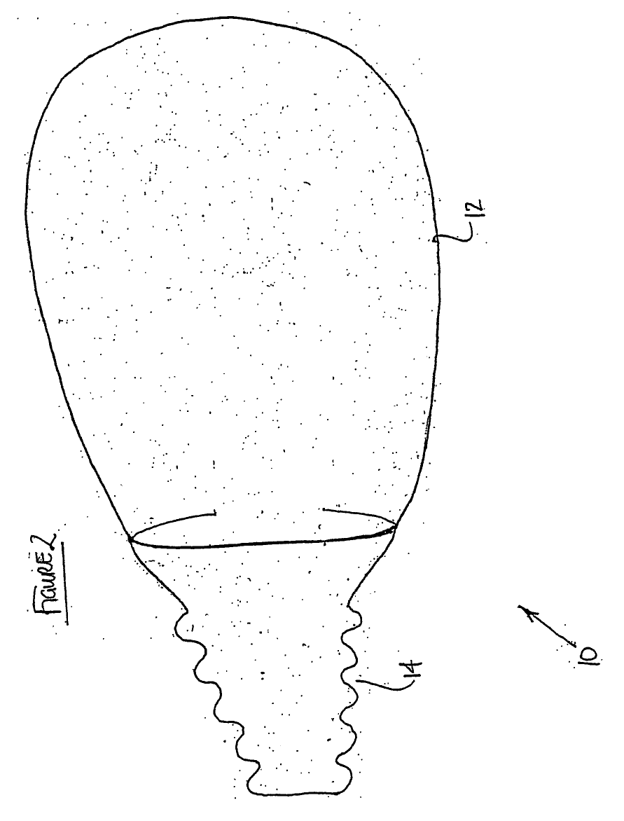

In an alternate embodiment, shown in FIG. 2, the tapered cuff preferably includes a number of spaced apart protrusions or “ribs”. The ribs serve a dual purpose.

Firstly, the ribs provide further strength to the cuff portion, particularly whilst the user is stretching the cuff to insert a limb into the device. That is, the ribs prevent the cuff from breaking or splitting while the cuff is in a stretched position (ie. as it is being fitted to the user). As a corollary, the extra strength provided by the ribs allow the cuff to be formed of a thinner material than the material which would normally be used in forming a device with a straight tapered profile. This preferably allows the device to be manufactured at a lower cost.

Secondly, the ribs serve as a visual guide, allowing the user to more easily perceive and select the correct region in which to cut or tear off a portion of the cuff, when selecting an appropriate size for the cuff.

Generally, a user would use a knife or a pair of scissors to select an appropriate length for the cuff. However, it will be understood that the cuff may also incorporate means for assisting the user in selecting the appropriate cuff size. For example, the cuff may include, at defined intervals, a series of “weakened zones”, which allow a user to tear away a portion of the cuff without the need for cutting implements such as knives or scissors.

The cuff may alternatively or additionally have pre-printed cut-lines to indicate appropriate cutting locations for a given user's limb size.

Typically the sleeve portion 12 is formed from a transparent polymer (such as polyethylene, polypropylene etc) to enable the user (and medical personnel) to maintain observation of the limb section enclosed therein.

The following paragraphs provide examples of an embodiment of the device in use on a patient (ie. after the cuff has been appropriately sized).

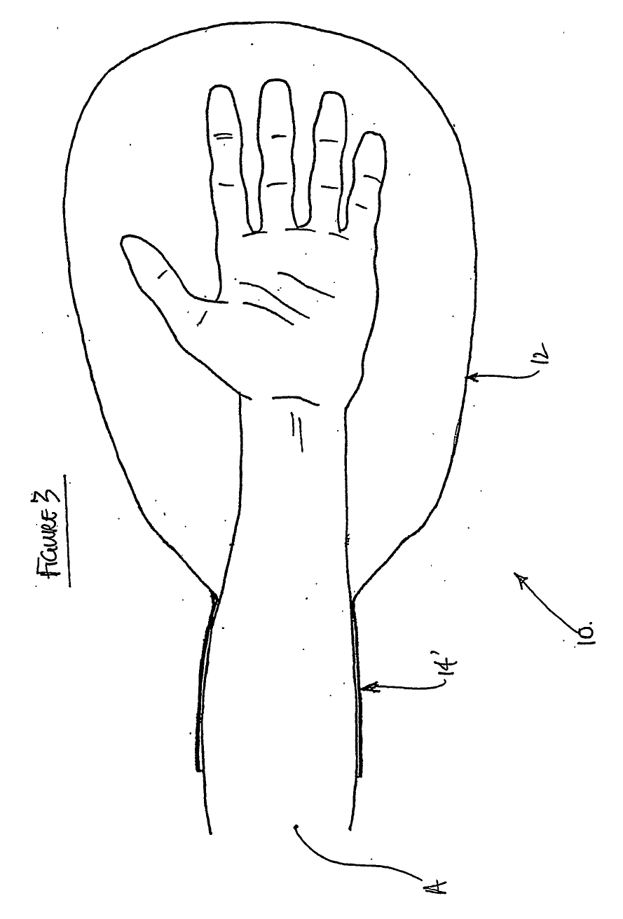

Referring now to FIG. 3, there is shown a bag typically adapted for enclosing the hand/wrist of a user. The cuff portion 14 has been cut to an appropriate size. The cuff portion 14 is defined simply by a continuation of the sleeve portion 12 (ie. is formed of the same material as the sleeve portion). Optionally, the cuff portion may have a thicker wall, or may be formed from a higher density version of the same material (eg. a low density polyethylene sleeve portion and a high density polyethylene cuff portion). Further, the cuff portion in FIG. 3 can be impregnated by an elastic material.

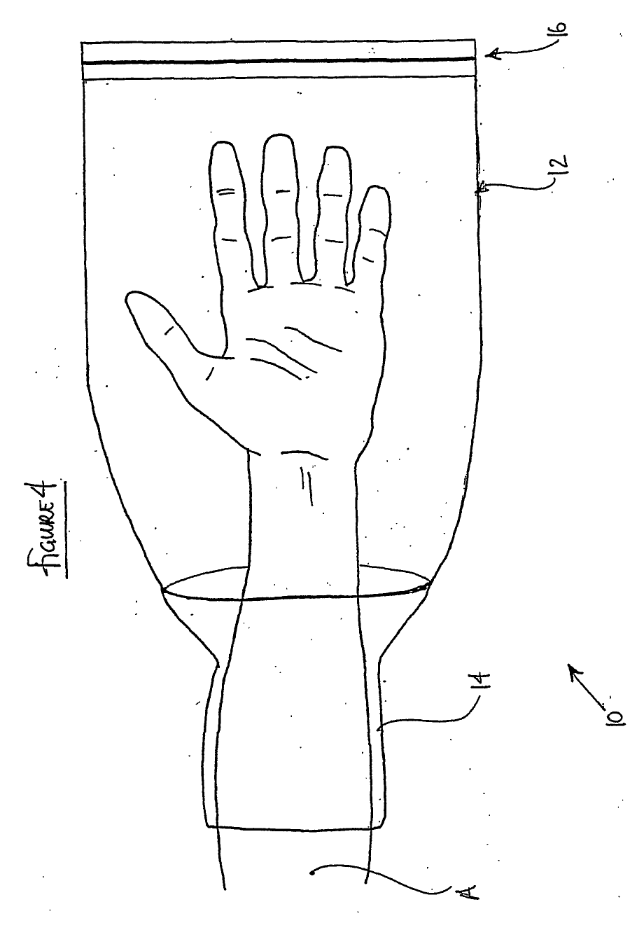

Referring now to FIG. 4, the end of the bag opposing the cuff portion may be resealably openable, for example by having a press-seal opening 16 formed thereat. Other types of resealable openings can also be employed, such as those employing resealable waterproof contact adhesives, etc.

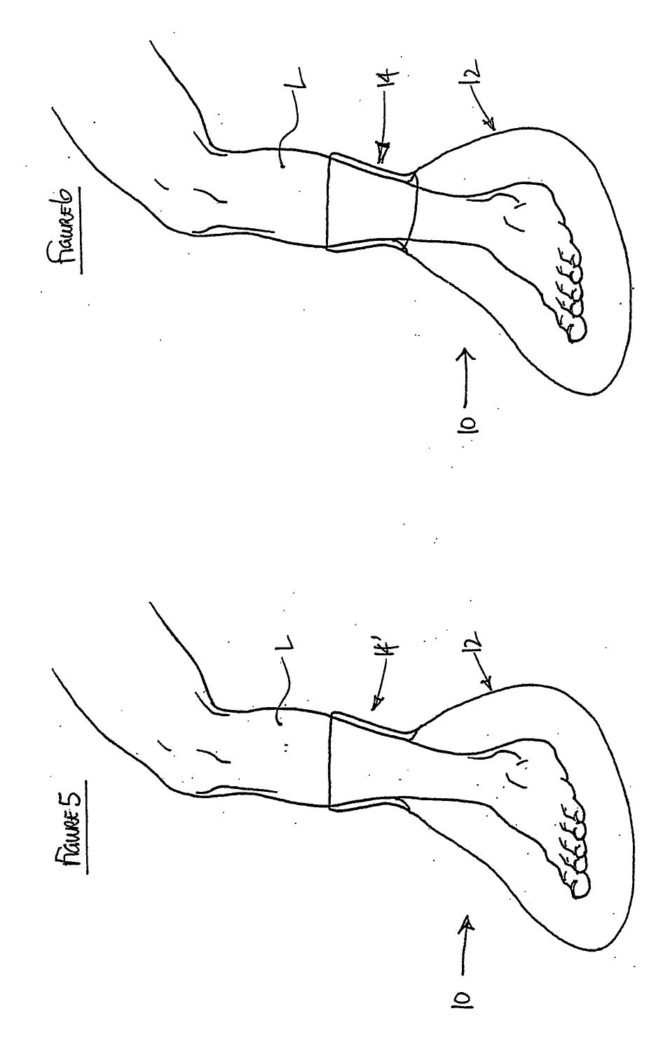

Referring now to FIGS. 5 and 6 where like reference numerals are used to denote similar or like parts, the cuff portion 14 surrounds a shin and calf of the user to enclose the foot/ankle of the user. Either the same protective bag can be used for both the hand/wrist and the foot/ankle, or the bag can be a different size for foot/ankle usage.

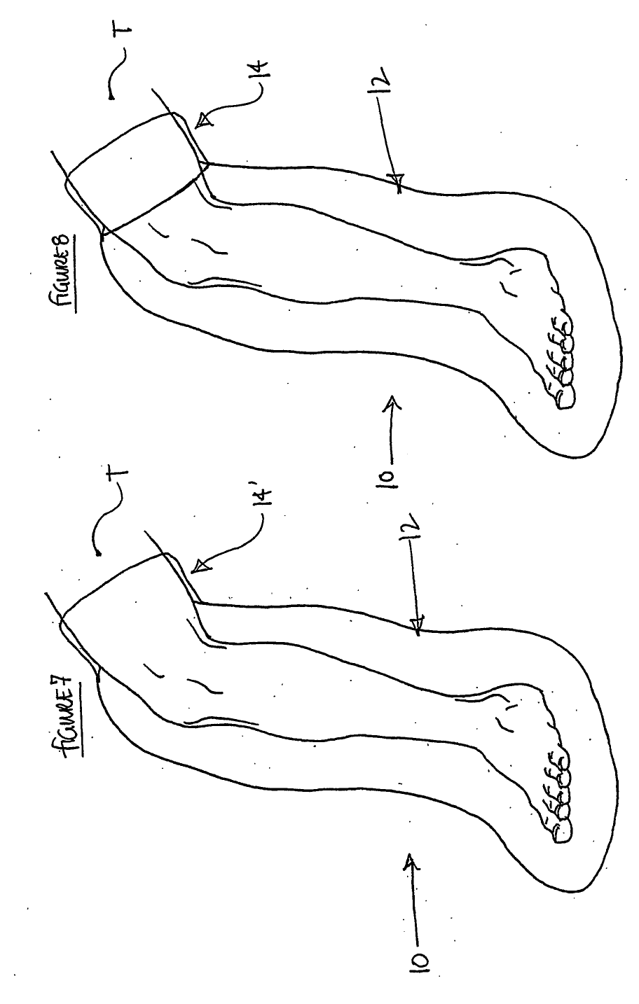

Referring now to FIGS. 7 and 8, again where like reference numerals are used to denote similar or like parts to that of FIGS. 5 and 6, the sleeve portion 12 is substantially elongate, to fit both the foot, lower leg, knee, and lower thigh region of a user therein. In addition, the tapered cuff portion is typically sized with a greater average diameter to fit around the thigh T of a user.

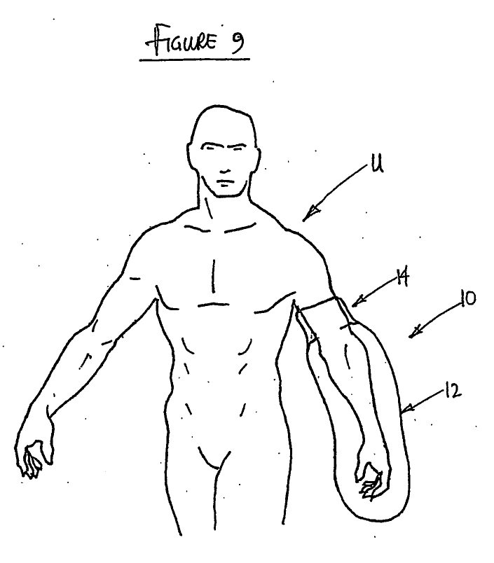

Referring now to FIG. 9, where like reference numerals are used to denote similar or like parts, a differently shaped and elongated sleeve portion 12 is shown which is adapted for enclosing the hand and forearm of a user U.

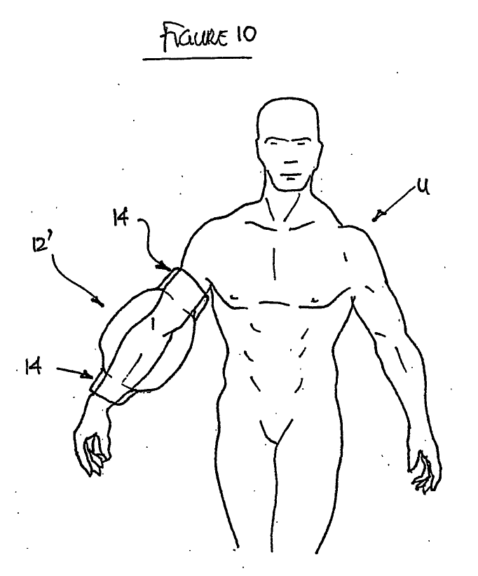

Referring now to FIG. 10, where like reference numerals are used to denote similar or like parts, a further modified sleeve portion 12′ includes a pair of opposing cuff portions 14. The upper cuff portion seals against the user's upper arm and the lower cuff portion seals against the forearm. The sleeve 12′ thus isolates the user's elbow region, whilst still providing for hand and arm mobility. A similar arrangement can be adapted for positioning around a user's knee region.

The embodiments described above most typically have medical applications, for protecting cuts, abrasions, burns, plasters, bandages etc against moisture, gas, contact, knocking and bumping. However, the arrangements can also be used in work and domestic applications wherever similar limb protection is required. The embodiments are particularly adapted for use by a user in showering and bathing applications.

Whilst the invention has been described with reference to a number of preferred embodiments, it should be appreciated that the invention can be embodied in many other forms.

1. A limb protection device for protecting a section of a user's limb, the device comprising:
an enclosure for enclosing the limb section; and
at least one flexible cuff integral with and away from which the enclosure extends, the cuff defining an opening into the device and having a tapered profile arranged to allow, in use, the selection of an appropriately sized opening into the device, whereby the appropriately sized opening in the cuff is selectable to stretch around and compress against that part of the user's limb extending therethrough, to prevent the ingress of matter into the device immediately at the device opening.

2. A device in accordance with claim 1, wherein the cuff further comprises a series of spaced-apart protrusions extending along the length of the cuff, wherein the protrusions provide structural integrity to the cuff.

3. A device in accordance with claim 2, wherein the protrusions are integrally moulded into the cuff.

4. A device in accordance with claim 2 or claim 3, wherein the protrusions are of a substantially semi-circular shape.

5. A device in accordance with any preceding claim, wherein the cuff has a width that is greater than a wall thickness of the enclosure.

6. A device in accordance with any one of the preceding claims, wherein the cuff is a continuation of the enclosure and is formed from the same material as the enclosure, or is formed from a different material, or a more dense form of the same material, as the enclosure.

7. A device in accordance with any one of the preceding claims, wherein the cuff is formed from a resilient polymeric material such as latex, or elastic impregnated plastic, to enable its stretching during fitting and removal of the device to a limb.

8. A device in accordance with any one of the preceding claims, wherein the enclosure is a sleeve that is either:
(a) closed at one end, with the cuff located at an opening at the other opposing end; or
(b) open at opposing ends, with respective cuffs located at each end.

9. A device in accordance with claim 8, wherein the sleeve in (a) is releasably openable at said one end for enabling access to the limb section when the device is fitted to a user.

10. A device in accordance with claim 9, wherein a press-seal opening is provided at said one end.

11. A device in accordance with any one of claims 8 to 10, wherein the sleeve in (a) is adapted for receiving a user's hand or foot therein; and the sleeve in (b) is adapted for receiving the limb right therethrough, with the cuff being located on opposing sides of the limb section that is enclosed within the enclosure.

12. A device in accordance with any one of the preceding claims, wherein the enclosure and/or the cuff are transparent.

13. A device in accordance with any one of the preceding claims, wherein the enclosure is formed from a polymer that is integrally moulded with the cuff.

14. A device in accordance with any one of the preceding claims, wherein a therapeutic agent is provided in the interior of the device.
